(19) United States
(12) Patent Application Publication
(10) Pub. No.: US 2022/0136281 A1
(43) Pub. Date: May 5, 2022

(54) Sanitizing Door Handle

(71) Applicant: KleanTouch LLC, Scottsdale, AZ (US)

(72) Inventors: Michael Rafko, Scottsdale, AZ (US); Natalie K. Myers, Myrtle Beach, SC (US); James Bell, White Plains, NY (US); Ian Carrus, Naperville, IL (US); Daniel W. Allen, Mesa, AZ (US); Joseph M. Barrett, Tempe, AZ (US); Joseph Pepe Elijio Velasquez, Fountain Hills, AZ (US)

(21) Appl. No.: 17/514,402

(22) Filed: Oct. 29, 2021

Related U.S. Application Data
(60) Provisional application No. 63/107,148, filed on Oct. 29, 2020.
(60) Provisional application No. 63/254,094, filed on Oct. 9, 2021.

(51) Int. Cl. E05B 1/00; A61L 2/00

(52) U.S. Cl. CPC E05B 1/0069; A61L 2202/15; A61L 2/0088

(57) ABSTRACT

Various implementations of the invention are directed toward a sanitizing door handle and/or components of such sanitizing door handle that sanitizes the door handle during use. Various implementations of the invention are directed toward a gravity-fed sanitizing door handle and/or components of such sanitizing door handle. Various implementations of the invention are directed to a hand sanitizing dispenser that dispenses a fluid, such as a sanitizing fluid, onto hand(s) of a user when the user operates or otherwise engages a door handle, such as, but not limited to, the sanitizing door handles discussed above. In some implementations of the invention, the fluid is dispensed directly onto the hands of the user. In some implementations of the invention, the fluid is dispensed through dispensers and onto the hands of the user through contact with the dispensers. In some implementations of the invention, the fluid is dispensed onto the exterior of the sanitizing door handle and is subsequently passed onto the hands of the user through contact with the sanitizing door handle.

### CROSS-REFERENCE TO RELATED APPLICATIONS

This application claims priority to U.S. Provisional Patent Application No. 63/107,148, entitled “Sanitizing Door Handle,” filed on Oct. 29, 2020; and this application claims priority to U.S. Provisional Patent Application No. 63/254,094, entitled “Sanitizing Door Handle,” filed on Oct. 9, 2021. Each of the foregoing patent applications is incorporated herein by reference in its entirety.

### FIELD OF THE INVENTION

The invention is generally related to door handles, and more specifically to sanitizing door handles.

### BACKGROUND OF THE INVENTION

Conventional door handles suffer from actual or perceived cleanliness or sanitation issues. Such door handles may include, but are not limited to, restroom door handles, medical/hospital door handles, refrigerator or other food storage door handles, hotel/motel door handles, airplane bathroom handles, school door handles, clean room door handles, and other door handles.

In some instances, door handles with improved cleanliness and/or sanitation are needed. In some instances, door handles with fluid dispensers, such as, but not limited to sanitizing fluid dispensers, are needed.

### SUMMARY OF THE INVENTION

Various implementations of the invention are directed toward a sanitizing door handle and/or components of such sanitizing door handle, such that the sanitizing door handle is sanitized or cleaned in some fashion. Various implementations of the invention are directed toward a manual sanitizing door handle and/or components of such sanitizing door handle. In various implementations of the invention, the fluid selected for use with various implementations of the invention controls, in part, the level of “cleaning” or “sanitizing” of the door handle from sterilized to disinfected to cleaned or to some other level of cleanliness.

Various implementations of the invention are directed to a hand sanitizing dispenser that dispenses a fluid, such as a sanitizing fluid, onto hand(s) of a user when the user operates or otherwise engages a door handle, such as, but not limited to, the sanitizing door handles discussed above. In some implementations of the invention, the fluid is dispensed directly onto the hands of the user. In some implementations of the invention, the fluid is dispensed onto the exterior of the sanitizing handle and is subsequently passed onto the hands of the user through contact with the sanitizing door handle. In various implementations of the invention, the fluid selected for use with various implementations of the invention controls, in part, the level of “cleaning” or “sanitizing” of the hands of the user from sterilized to disinfected to cleaned or to some other level of cleanliness.

### BRIEF DESCRIPTION OF THE DRAWINGS

FIGS. 1A and 1B illustrate a sanitizing door handle from a first perspective and a second perspective, respectively, according to various implementations of the invention.

FIG. 2 illustrates a dispenser unit that may be used in various implementations of the invention.

FIG. 3 illustrates a plurality of dispensers implemented in a sanitizing door handle according to various implementations of the invention.

FIG. 4 illustrates a plurality of dispensers implemented in a sanitizing door handle according to various implementations of the invention.

FIG. 5 illustrates a plurality of dispensers disposed in a distribution manifold, which in turn is disposed in a sanitizing door handle according to various implementations of the invention.

FIG. 6 illustrates a plurality of dispensers implemented in a sanitizing door handle according to various implementations of the invention.

FIGS. 7A and 7B illustrate a plurality of dispensers implemented on a sanitizing door handle from a first perspective and a second perspective, respectively, according to various implementations of the invention.

FIG. 8 illustrates a plurality of dispensers implemented in a sanitizing door handle according to various implementations of the invention.

FIG. 9 illustrates a distribution manifold for a sanitizing door handle according to various implementations of the invention.

FIG. 10 illustrates a sanitizing door handle according to various implementations of the invention.

FIG. 11 illustrates a replaceable refill unit according to various implementations of the invention.

FIG. 12 illustrates an interior of a sanitizing door handle according to various implementations of the invention.

FIG. 13 illustrates a plurality of dispensers under a single permeable membrane implemented in a sanitizing door handle according to various implementations of the invention.

FIG. 14 depicts a sanitizing door handle configured for parallel use with an existing door handle according to various implementations of the invention.

FIG. 15 depicts a sanitizing door handle adapter configured to replace an existing door handle according to various implementations of the invention.

FIG. 16 depicts a sanitizing door handle according to various implementations of the invention.

FIG. 17 depicts a sanitizing door handle according to various implementations of the invention.

FIG. 18 depicts a replaceable refill unit configured to use with a sanitizing door handle according to various implementations of the invention.

FIGS. 19A and 19B depict sanitizing door handles according to various implementations of the invention.

FIG. 20 depicts a sanitizing door handle cassette according to various implementation of the invention.

FIG. 21A depicts a sanitizing door handle cassette installed on a door handle according to various implementations of the invention, and FIGS. 21B-21C depict different adaptors for installing a sanitizing door handle cassette according to various implementations of the invention.

FIG. 22 depicts a sanitizing door handle according to various implementations of the invention.

FIG. 23 depicts a distribution manifold partially installed in a door handle housing according to various implementations of the invention.

FIG. 24 depicts a portion of a distribution manifold according to various implementations of the invention.

FIG. 25 depicts dispensing applicators with perforated plastic liquid permeable membranes according to various implementations of the invention.

FIG. 26 illustrates a dispenser unit that may be used in various implementations of the invention

### DETAILED DESCRIPTION

Various implementations of the invention are directed toward a sanitizing door handle and/or components of such sanitizing door handle such that the sanitizing door handle is sanitized or cleaned in some fashion. Various implementations of the invention are directed toward a manual sanitizing door handle and/or components of such sanitizing door handle. In such implementations, a user manually operates door handle that dispenses a fluid, such as, but not limited to, a sanitizing fluid, onto a grip of the sanitizing door handle during operation of the door handle. In various implementations of the invention, the fluid selected for use with various implementations of the invention controls, in part, the level of “cleaning” or “sanitizing” of the door handle from sterilized to disinfected to cleaned or to some other level of cleanliness.

Various implementations of the invention are directed to a hand sanitizing dispenser that dispenses a fluid, such as a sanitizing fluid, onto hand(s) of a user when the user operates or otherwise engages a door handle, such as, but not limited to, the sanitizing door handles discussed above. In some implementations of the invention, the fluid is dispensed directly onto the hands of the user. In some implementations of the invention, the fluid is dispensed onto the exterior of the sanitizing handle and is subsequently passed onto the hands of the user through contact with the sanitizing door handle. In various implementations of the invention, the fluid selected for use with various implementations of the invention controls, in part, the level of “cleaning” or “sanitizing” of the hands of the user from sterilized to disinfected to cleaned or to some other level of cleanliness.

Various implementations of the invention are directed to a gravity-fed hand sanitizing dispenser that dispenses a fluid, such as a sanitizing fluid, onto a door handle and/or hand(s) of a user when the user operates or otherwise engages the door handle, such as, but not limited to, the sanitizing door handles discussed above.

In some implementations of the invention, the fluid may be a sanitizing fluid that includes any fluid having sterilizing, disinfecting, antiseptic, antimicrobial, and/or other sanitizing or cleaning properties. Active ingredients in the sanitizing fluid may include alcohol-based sanitizing fluids such as, but not limited to, isopropyl alcohol, ethyl alcohol, n-proponal, and other alcohol-based fluids; or non-alcohol-based sanitizing fluids such as, but not limited to, benzalkonium chloride (a chemical disinfectant), triclosan (an antiseptic), thymol (an organic antibacterial agent), and/or other non-alcohol-based sanitizing fluids. In some implementations of the invention, the active ingredients may be mixed with a carrier ingredient such as glycerol or other gel-like ingredient to achieve a certain amount of viscosity as would be appreciated. In some implementations of the invention, the active ingredients may be aerosolized to be sprayed onto hand(s) or door handles as would be appreciated. Sanitizing fluids are available from various manufacturers and sold under names, including, but not limited to: Purell®, Germ-X®, CleanWell™, Babyganics®, CVS®, BlumNaturals®, and other manufacturers and/or names.

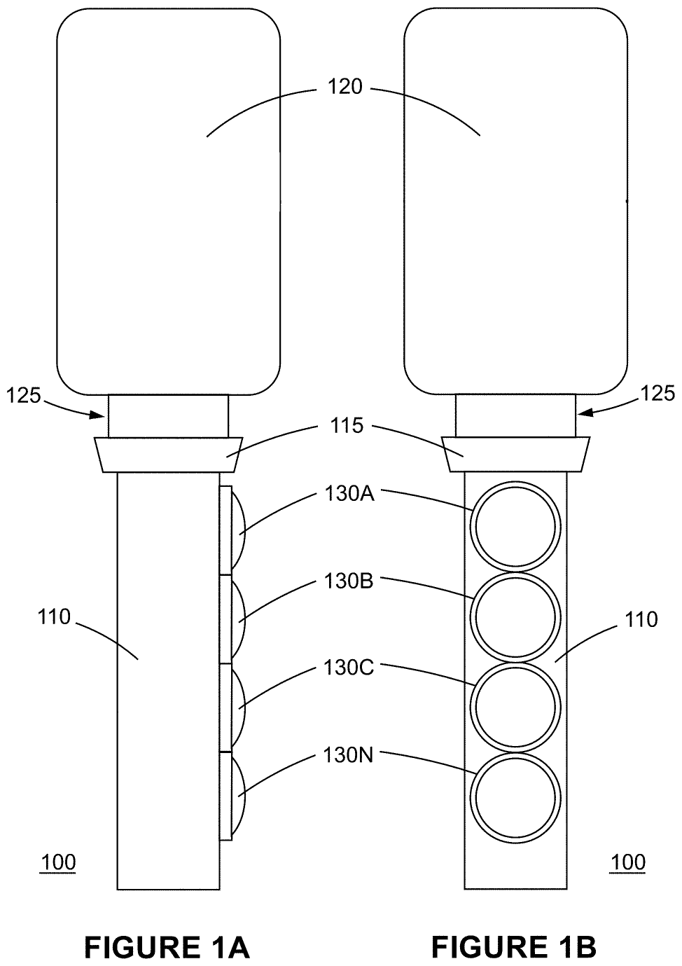

FIG. 1A illustrates a sanitizing door handle 100 from a first perspective and FIG. 1B illustrates sanitizing door handle 100 from a second perspective, according to various implementations of the invention. In some implementations of the invention, door handle 100 is a gravity-fed sanitizing door handle, though in other implementations, door handle 100 may employ a pump. Door handle 100 includes a door handle housing 110, a fluid container 120, and a fluid container coupler 115. In some implementations of the invention, fluid container 120 may include a fluid container neck 125. In some implementations of the invention, door handle housing 110 may be fabricated from a variety of materials, including, but not limited to: metal, plastic, ceramic, fiberglass, wood, polymer, rubber, or other materials that may be used to fabricate door handle housing 110, and/or combinations thereof, as would be appreciated. In some implementations of the invention, door handle housing 110 forms a substantially hollow interior housing chamber (not illustrated in FIG. 1). In some implementations, door handle housing 110 may be formed from one or more door housing components. In some implementations, door handle housing 110 may also be formed to house fluid container 120. In some implementations of the invention, at least one door housing component is movable and/or detachable from door handle housing 110 in order to provide access to interior housing chamber. In some implementations of the invention, door handle housing 110 is detachable from a door in order to provide access to interior housing chamber. Interior housing chamber and its purpose is described in further detail below.

Door handle 100 may be configured as a grip, a knob, a lever, a button, a pushbar, a pullbar, a latch, or other suitable handle by which a user opens, closes, latches, unlatches, locks, unlocks, activates, or otherwise operates door handle 100 to open or close a door to which door handle 100 is operatively attached. In some implementations of the invention, door handle 100 may be configured to push open the door. In some implementations of the invention, door handle 100 may be configured to pull open the door. In some implementations of the invention, door handle 100 may be configured to push to actuate a latch to open the door. In some implementations of the invention, door handle 100 may be configured to pull to actuate the latch to open the door. In some implementations of the invention, door handle 100 may be configured to rotate up to open the door. In some implementations of the invention, door handle 100 may be configured to rotate down to open the door. In some implementations of the invention, door handle 100 may be configured to rotate up to actuate the latch to open the door. In some implementations of the invention, door handle 100 may be configured to rotate down to actuate the latch to open the door. In some implementations of the invention, door handle 100 may be configured to slide left to open the door. In some implementations of the invention, door handle 100 may be configured to slide right to open the door. In some implementations of the invention, door handle 100 may be configured to slide left to actuate the latch to open the door. In some implementations of the invention, door handle 100 may be configured to slide right to actuate the latch to open the door. In some implementations of the invention, door handle 100 may be configured to be depressed to open the door. In some implementations of the invention, door handle 100 may be configured to be depressed to actuate the latch to open the door. In some implementations of the invention, door handle 100 may be configured to open the door or actuate the latch in other manners as would be appreciated.

According to various implementations of the invention, door handle 100 includes a number of sanitizing liquid applicating dispensers 130 (illustrated as a dispenser 130A, a dispenser 130B, a dispenser 130C, . . . and a dispenser 130N). In some implementations, dispensers 130 are formed into door handle housing 110; in some implementations, dispensers 130 are individual standalone sub-components that snap-fit, or otherwise couple into door handle housing 110; in some implementations, dispensers 130 are disposed in a manifold (not otherwise illustrated in FIG. 1) that in turn snap-fit, slide-fit, or otherwise couple onto or into door handle housing 110. According to various implementations of the invention, dispensers 130 comprise a liquid permeable, or semi-permeable membrane that when compressed (i.e., pressured applied), releases a flow of the fluid to and through the membrane, and subsequently, into contact with door handle housing 110 and/or a user's hand.

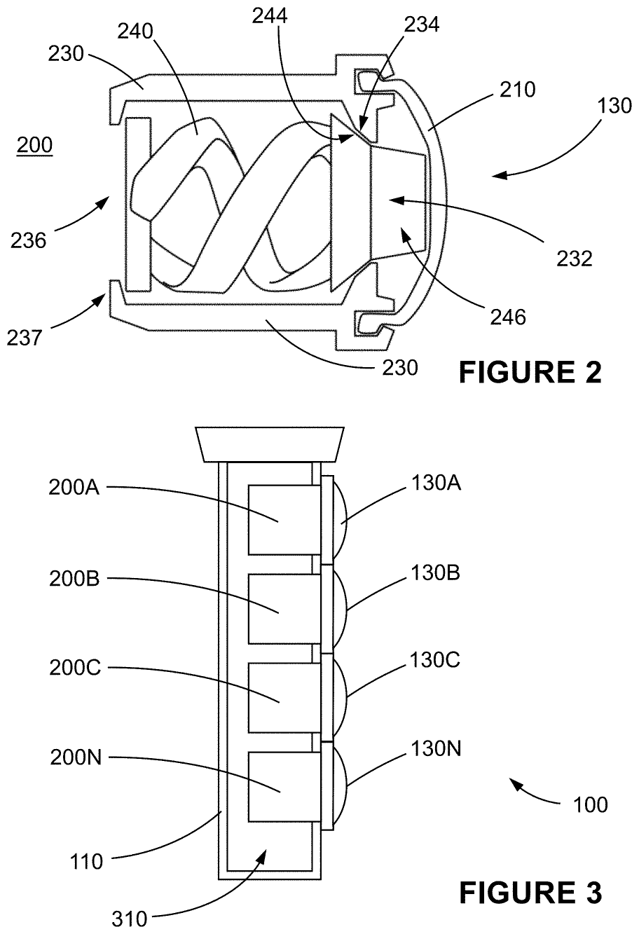

FIG. 2 illustrates a standalone dispenser unit 200 (i.e., individual standalone sub-component liquid applicating dispenser 130 referenced above) that may be used in various implementations of the invention. The description of various aspects of dispenser unit 200 may apply to other dispensers 130 that may be used in other implementations as would be appreciated. In some implementations of the invention, dispenser unit 200 includes a liquid permeable membrane 210. In some implementations, the membrane may be a porous material. In some implementations, the membrane may be a cloth-like material. In some implementations, the membrane may be a sponge-like material. In some implementations, the membrane may be a non-porous, or semi-porous, material with perforations. In some implementations, the membrane may be a plastic material with perforations.

In some implementations of the invention, liquid permeable membrane 210 may include an outer liquid permeable membrane and an inner liquid permeable membrane (not separately illustrated). In some implementations, the outer membrane may be a porous material. In some implementations, the outer membrane may be a cloth-like material. In some implementations, the outer membrane may be a sponge-like material. In some implementations, the outer membrane may be a non-porous, or semi-porous, material with perforations. In some implementations, the outer membrane may be a plastic material with perforations. In some implementations, the inner membrane may be a porous material. In some implementations, the inner membrane may be a cloth-like material. In some implementations, the inner membrane may be a sponge-like material. In some implementations, the outer membrane may be protective material and the inner membrane may be an absorbent material. In some implementations, the outer membrane may be cloth-like material and the inner membrane may be a sponge-like material. In some implementations, the inner membrane and/or the outer membrane may be other permeable membrane materials as would be appreciated. Various other combinations of the inner and outer membranes described may be used as would be appreciated.

In some implementations of the invention, dispenser unit 200 includes a dispenser housing 230 and a dispenser spring 240 disposed inside dispenser housing 230. In some implementations of the invention, dispenser spring 240 includes a spring head 246 with a spring interface 244. In some implementations of the invention, dispenser housing 230 includes an output opening 232, an input opening 236, a spring seat 234, and a spring retainer 237. In some implementations of the invention, fluid is received into an interior of dispenser housing 230 via input opening 236. In some implementations of the invention, fluid is dispensed out of dispenser housing 230 via output opening 232. In some implementations of the invention, spring head 246 prevents fluid from being dispensed out of output opening 232 of dispenser housing 230; in this closed “default” position, spring 240 resides in a compressed state between spring retainer 237 and spring seat 234. In some implementations of the invention, spring interface 244 presses against spring seat 234 to prevent fluid from dispensing out of output opening 232. In some implementations of the invention, when spring 240 is compressed (e.g., by a user pressing spring head 246 through membrane 210), spring interface 244 disengages from spring seat 234 to allow fluid to dispense out of output opening 232.

While the figures illustrate dispenser 130 and dispenser unit 200 as having a circular shape, various implementations of the invention may use other shapes and/or sizes (or multiple different shapes and/or sizes), for example, depending upon the nature, shape and/or size of door handle 100, or other factors, as would be appreciated. Also, while the figures illustrate multiple dispensers 130 or multiple dispenser units 200, various implementations may use more or fewer numbers of such dispensers/dispenser units, including a single dispenser/dispenser unit, for example, depending upon the nature, shape and/or size of door handle 100, or other factors, as would be appreciated.

In some implementations of the invention, dispenser unit 200 may be an off-the-self product commercially marketed and sold as a “Dab-O-Matic Applicator” from Dabomatic Corporation, Mount Vernon, N.Y., www.dabomatic.com.

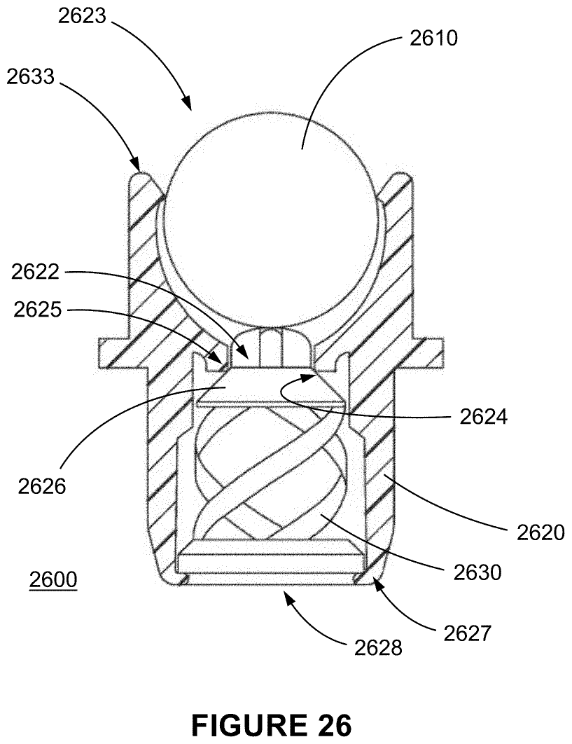

FIG. 26 illustrates a standalone dispenser unit 2600 (i.e., individual standalone sub-component liquid applicating dispenser 130 referenced above) that may be used in various implementations of the invention. The description of various aspects of dispenser unit 2600 may apply to other dispensers 130 that may be used in other implementations as would be appreciated. In some implementations of the invention, dispenser unit 2600 includes a push ball 2610 and a dispenser spring 2620 disposed inside a dispenser housing 2630. In some implementations of the invention, dispenser spring 2620 includes a spring head 2626 with a spring interface 2624. In some implementations of the invention, dispenser housing 2620 includes a spring output opening 2622, a push ball output opening 2623, an input opening 2628, a spring seat 2625, and a spring retainer 2627. In some implementations of the invention, fluid is received into an interior of dispenser housing 2620 via input opening 2628. In some implementations of the invention, fluid is dispensed out of dispenser housing 2620 through push ball output opening 2623 via spring output opening 2622.

In some implementations of the invention, spring head 2626 prevents fluid from being dispensed out of spring output opening 2622 of dispenser housing 2620 and push ball 2610 prevents fluid from being dispensed out of push ball output opening 2623; in this closed “default” position, spring 2620 resides in a compressed state between spring retainer 2627 and spring seat 2625. In some implementations of the invention, spring interface 2624 presses against spring seat 2625 to prevent fluid from dispensing out of spring output opening 2622. In some implementations of the invention, spring 2620 presses push ball 2610 against a push ball retainer 2633 to prevent fluid from dispensing out of push ball output opening 2623. In some implementations of the invention, when push ball 2610 is pressed, thereby compressing spring 2620 (e.g., by a user pressing push ball 2610), spring interface 2624 disengages from spring seat 2625 and/or push ball 2610 disengages from push ball retainer 2633 to allow fluid to dispense out of spring output opening 2622 and/or push ball output opening 2623.

In some implementations of the invention, dispenser unit 2600 may include an optional membrane or other covering over push ball 2610, similar to those described above with regard to membrane 210 as would be appreciated (not otherwise illustrated in FIG. 26).

In some implementations of the invention, dispenser unit 2600 may also be an off-the-self product commercially marketed and sold by Dabomatic Corporation. Other dispenser units from this and other companies may be similarly utilized and/or configured for use with various implementations of the invention as would be appreciated. Various implementations of the invention are described with reference to dispenser unit 200; however, dispenser unit 2600, or any other dispenser, may be used in place of or conjunction with dispenser unit 200 as would be appreciated.

While the figures illustrate dispenser 130 as having a circular shape, various implementations of the invention may use other shapes and/or sizes (or multiple different shapes and/or sizes), for example, depending upon the nature, shape and/or size of door handle 100, or other factors, as would be appreciated. Also, while the figures illustrate multiple dispensers 130, various implementations may use more or fewer numbers of such dispensers/dispenser units, including a single dispenser/dispenser unit, for example, depending upon the nature, shape and/or size of door handle 100, or other factors, as would be appreciated.

FIG. 3 illustrates a plurality of dispensers 130, each comprising a dispenser unit 200 (illustrated as a dispenser unit 200A, a dispenser unit 200B, a dispenser unit 200C, and a dispenser unit 200N) in door handle 100 according to various implementations of the invention. In some implementations of the invention, door housing 110 of door handle 100 forms an interior chamber 310 which receives fluid from fluid container 120 (of FIG. 1). In some implementations of the invention, each dispenser unit 200 receives fluid from interior chamber 310 through, for example, input opening 236 (illustrated in FIG. 2) and subsequently dispenses fluid through output opening 232 (illustrated in FIG. 2) when spring 240 is further compressed.

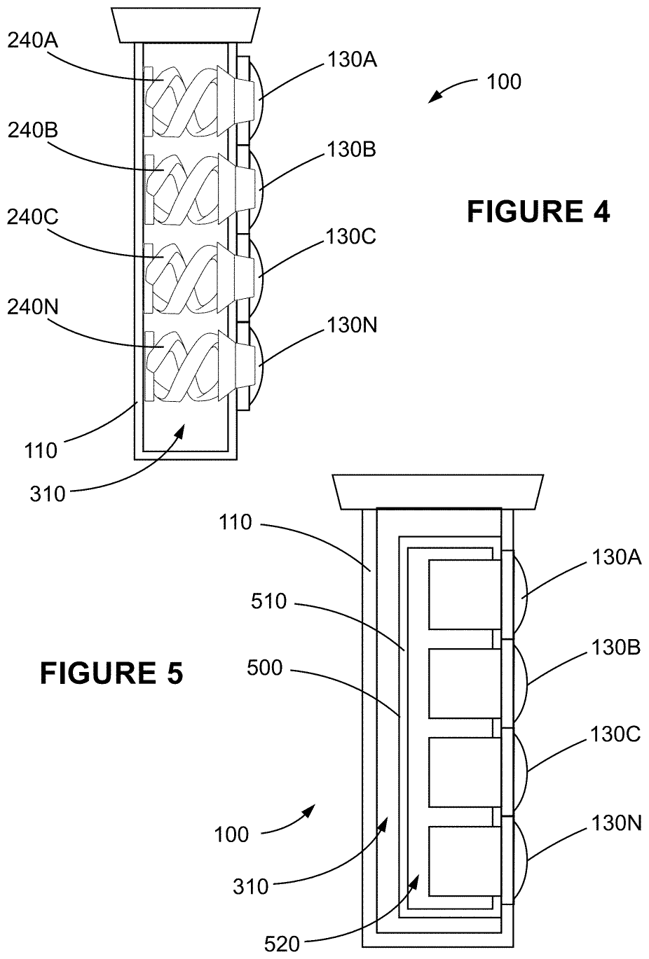

FIG. 4 illustrates a plurality of dispensers 130 implemented in door handle 100 according to various implementations of the invention. In such implementations, door handle housing 110 may be configured to replace and/or provide various aspects and/or functionality of dispenser housing 230 of FIG. 2. In particular, a portion of door handle housing 110 (e.g., a left wall of door handle housing 110 in FIG. 4) provides the functionality of spring retainer 237 and output opening 232 and spring seat 234 may be formed from and into door handle housing 110 to provide similar operation of springs 240 (illustrated as a spring 240A, a spring 240B, a spring 240C, . . . and a spring 240N) as would be appreciated. In some implementations of the invention, door handle housing 110 of door handle 100 forms interior chamber 310 which receives fluid from fluid container 120 and subsequently dispenses fluid through an output opening formed in door handle housing 110 when dispensers 130 are engaged by a user and spring(s) 240 are further compressed. As would be appreciated, door hand housing 110 may be similarly configured to replace and/or provide various aspects and/or functionality of dispenser housing 2620 of FIG. 26.

FIG. 5 illustrates a plurality of dispensers 130, each implemented as a dispenser unit 200, disposed in a distribution manifold 500, which in turn is disposed in door handle 100 according to various implementations of the invention. More particularly, distribution manifold 500 is disposed in interior chamber 310 formed by door handle housing 110 of door handle 100 according to various implementations of the invention. In some implementations of the invention, distribution manifold 500 is removably and/or replaceably disposed in interior chamber 310. In some implementations of the invention, distribution manifold 500 includes a manifold wall 510 which forms an interior manifold chamber 520. In some implementations of the invention, interior manifold chamber 520 receives fluid from fluid container 120 as would be appreciated and dispenses fluid from manifold chamber 520 through input opening(s) 235 and output opening(s) 232 of dispense unit(s) 200 as described above.

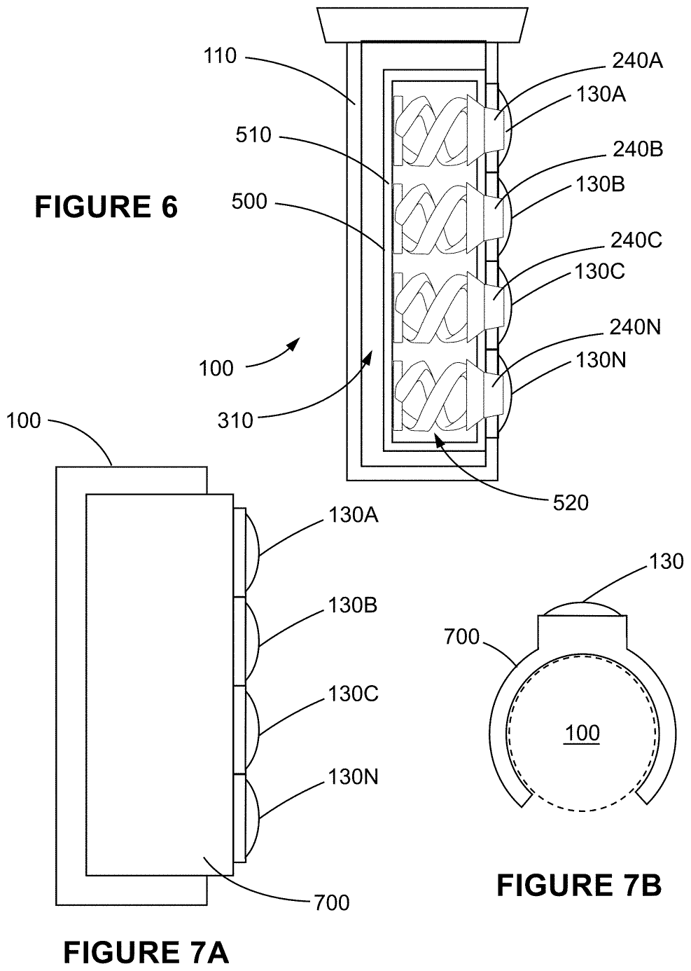

FIG. 6 illustrates a plurality of dispensers 130, each implemented as spring 240 disposed in distribution manifold 500, which in turn is disposed in interior chamber 310 formed by door handle housing 110 of door handle 100 according to various implementations of the invention. In some implementations of the invention, distribution manifold 500 is removably and/or replaceably disposed in interior chamber 310. In some implementations of the invention, distribution manifold 500 includes manifold wall 510 which forms interior manifold chamber 520. In some implementations of the invention, interior manifold chamber 520 receives fluid from fluid container 120 as would be appreciated and dispenses the fluid from manifold chamber 520 through dispensers 130 as described above with regard to FIG. 4.

FIG. 7A illustrates a manifold sleeve 700 according to a first perspective; and FIG. 7B illustrates manifold sleeve 700 according to a second perspective, each according to various implementations of the invention. In some implementations of the invention, manifold sleeve 700 attaches to an exterior of door handle 100 (as illustrated in FIG. 7B). Manifold sleeve 700 may snap fit, slide fit, couple, affix, adhere, velco, or otherwise secure to an exterior of door handle 100 in a variety of ways as would be appreciated. In various implementations of the invention, manifold sleeve 700 provides various features of the invention without significant modifications to door handle 100. Such implementations may include manifold sleeve 700, fluid container 120, and a flexible coupler (not illustrated in FIG. 7) between the two, where fluid container 120 is attached to the door, typically near door handle 100.

Such implementations accommodate a distribution manifold on an exterior of door handle 100, rather than in an interior of door handle 100 as would be appreciated.

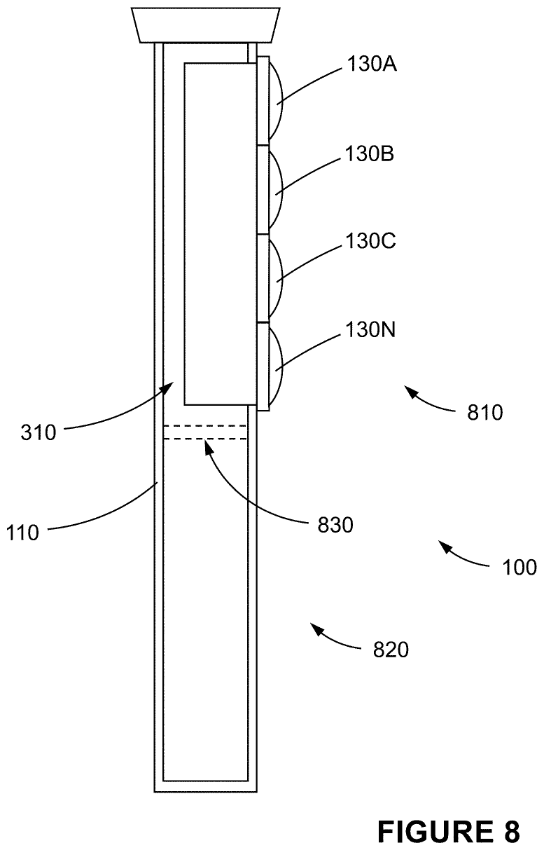

FIG. 8 illustrates a plurality of dispensers 130 disposed in door handle 100 in accordance with various implementations of the invention. In some implementations of the invention, an optional interior wall 830 separates door handle housing 110 into two portions: a first handle portion 810 and a second handle portion 820. Interior wall 830 restricts fluid to an interior of first handle portion 810 and not to an interior of second handle portion 820, thereby restricting fluid to the portion of door handle 100 in which dispensers 130 are disposed. In such implementations, a user engaging first handle portion 810 would contact dispenser(s) 130 and thus, fluid, whereas a user engaging second handle portion 820 would not contact dispenser(s) 130 and may not necessarily contact fluid as would be appreciated.

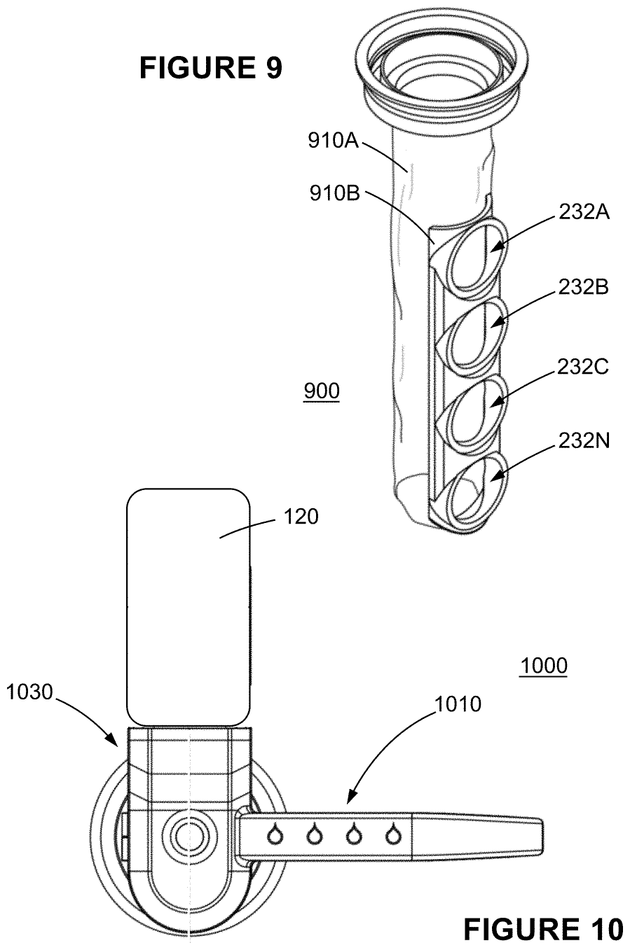

FIG. 9 illustrates a distribution manifold 900 according to various implementations of the invention. In some implementations of the invention, distribution manifold 900 comprises a flexible manifold wall portion 910A and a substantially rigid manifold wall portion 910B. As would be appreciated, substantially rigid manifold wall portion 910B is configured to accommodate dispensers 130 (or dispenser units 200) in output openings 232.

FIG. 10 illustrates a door handle 1000 according to various implementations of the invention. In some implementations of the invention, door handle 1000 includes a fluid container 120, a door latch mechanism 1030, and a door handle housing 1010. In some implementations, fluid container 120 stores a fluid, such as, but not limited to, a sanitizing fluid, as discussed above, and feeds a distribution manifold and associated dispensers (not otherwise illustrated in FIG. 10) housed in door handle housing 1010. As illustrated, door handle housing 1010 includes two portions: a first handle portion 1010A and a second handle portion 1010B. According to various implementations, first handle portion 1010A houses the distribution manifold and associated dispensers (not otherwise illustrated in FIG. 10), whereas, second handle portion 1010B does not. The distribution manifold in door handle housing 1010 restricts fluid to first handle portion 1010A and not to second handle portion 1010B, thereby restricting fluid to the portion of door handle 100 in which dispensers 130 are disposed. In such implementations, a user engaging first handle portion 1010A would contact dispenser(s) 130 and thus, fluid, whereas a user engaging second handle portion 1010B would not contact dispenser(s) 130 and may not necessarily contact fluid as would be appreciated.

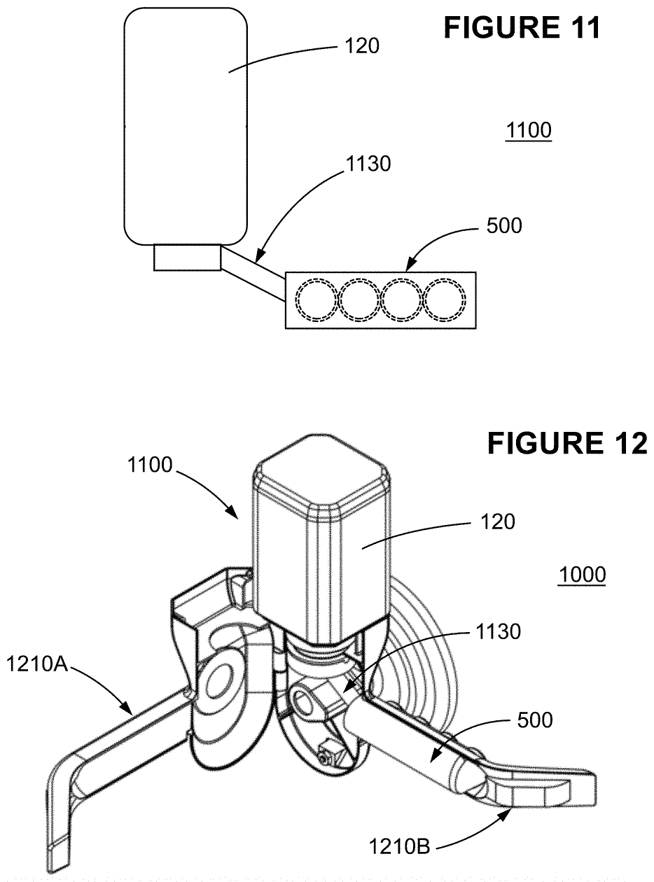

FIG. 11 illustrates a replaceable refill unit 1100 according to various implementations of the invention. According to various implementations, replaceable refill unit 1100 comprises a fluid container 120 with fluid, a coupler 1130, and a distribution manifold 500 with dispensers 130 (as illustrated as dashed lines due to being obscured by distribution manifold 500). According to various implementations, replaceable refill unit 1100 consists essentially of fluid container 120 with fluid, coupler 1130, and distribution manifold 500 with dispensers 130. According to various implementations of the invention, replaceable refill unit 1100 may be replaced in/on door handle 100 from time to time, as fluid is dispensed and used in connection with door handle 1000. In some implementations of the invention, replaceable refill unit 1100 consists essentially of fluid container 120 with fluid and distribution manifold 500 with dispensers 130; where coupler 1130 is configured for reuse as would be appreciated. In some implementations of the invention, replaceable refill unit 1100 consists essentially of fluid container 120 with fluid and a plurality of dispensers 130, including, but not limited to dispenser unit 200; where coupler 1130 and distribution manifold 500 are configured for reuse as would be appreciated. In some implementations of the invention, replaceable refill unit 1100 consists essentially of distribution manifold 500 with dispensers 130; where coupler 1130 is configured for reuse and where fluid container 120 is configured for refilling (as opposed to replacing) as would be appreciated. In some implementations of the invention, replaceable refill unit 1100 consists essentially of dispensers 130 or dispenser units 200; where distribution manifold 500 and coupler 1130 are configured for reuse and where fluid container 120 is configured for refilling (as opposed to replacing) as would be appreciated.

While fluid container 120 and distribution manifold 500 are depicted in a perpendicular arrangement (fluid container 120 in a vertical orientation and distribution manifold 500 in a horizontal orientation), fluid container 120 and distribution manifold 500 may be arranged in any other configuration depending on type of door handle 100 or application as would be appreciated. As would also be appreciated, coupler 1130 may be configured differently to accommodate other such configurations as would be appreciated. In some implementations, coupler 1130 may be formed of flexible tubing to accommodate installation with a variety of door handles 100; in some implementations, coupler 1130 may be formed from injection molded material to accommodate installation with specific door handle(s) 100; or in some implementations, coupler 1130 may be formed from other materials or other forming processes as would be appreciated.

FIG. 12 illustrates an interior of sanitizing door handle 1000 according to various implementations of the invention. In some implementations of the invention, door handle housing 1210 (illustrated in FIG. 12 as a housing portion 1210A and a housing portion 1210B) may open to reveal the interior of sanitizing door handle 1000 and to provide access to replaceable refill unit 1100. In some implementations of the invention, once door handle housing 1210 is open, replaceable refill unit 1100 (including fluid container 120, coupler 1130, and/or distribution manifold 500 with dispensers 130) may be replaced with a new replaceable refill unit 1100, or various components thereof, as would be appreciated. In some implementations of the invention, door handle housing 1210 may open for purposes of installing sanitizing door handle 1000 onto an existing door latch mechanism as would be appreciated.

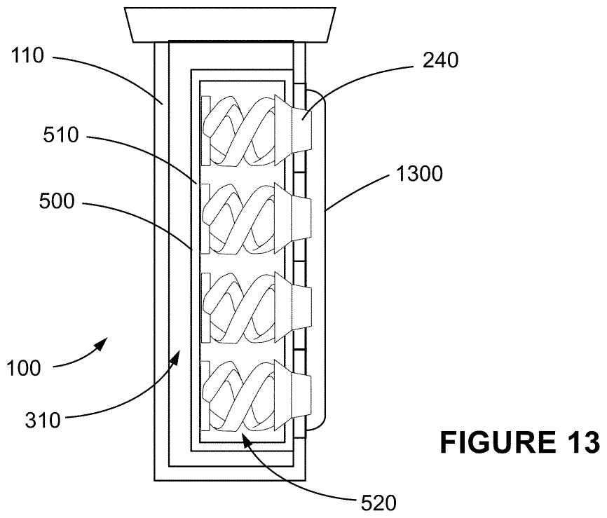

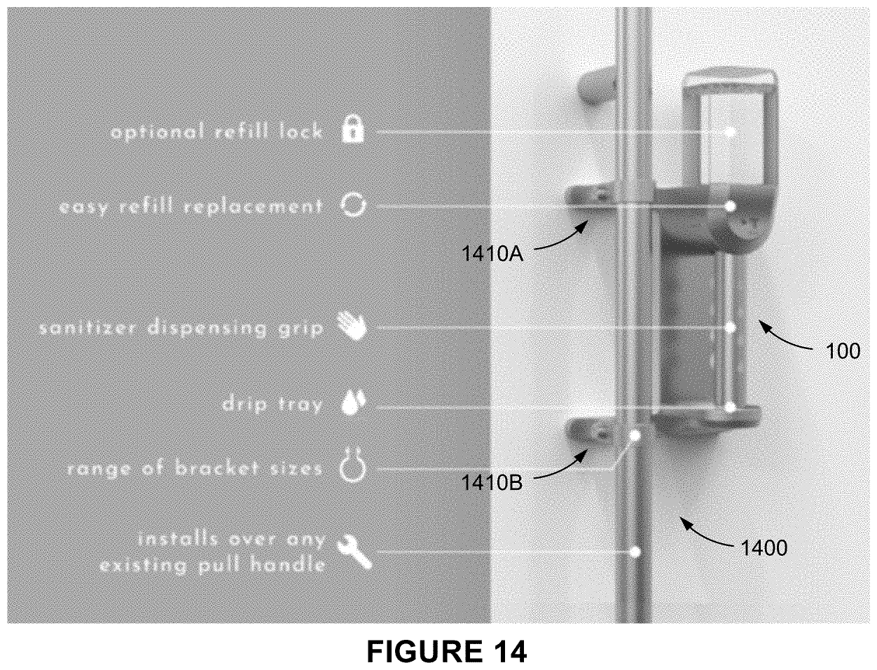

FIG. 13 illustrates a plurality of dispensers 130, each implemented as spring 240 disposed in distribution manifold 500, which in turn is disposed in interior chamber 310 formed by door handle housing 110 of door handle 100 similar to that of FIG. 6. However as illustrated in FIG. 13, springs 240 are configured under a single permeable membrane to dispense the fluid from manifold chamber 520 according to various implementations of the invention.

FIG. 14 depicts a sanitizing door handle configured for parallel use with an existing door handle according to various implementations of the invention. As depicted in FIG. 14, sanitizing door handle 100 may be configured to mount to an existing door handle 1400 by one or more brackets 1410 (illustrated as a bracket 1410A and a bracket 1410B). In this configuration, a user may use either sanitizing door handle 100 or existing door handle 1400 to open the door. In some implementations of the invention, bracket(s) 1410 may be adjustable or vary in size to fit a range of existing door handles 1400. While sanitizing door handle 100 is depicted as being mounted on a right side of existing door handle, sanitizing door handle 100 may be configured to mount on a left side, or either side, of existing door handle according to some implementations of the invention. Similar implementations may apply to existing horizontal door handles as would be appreciated.

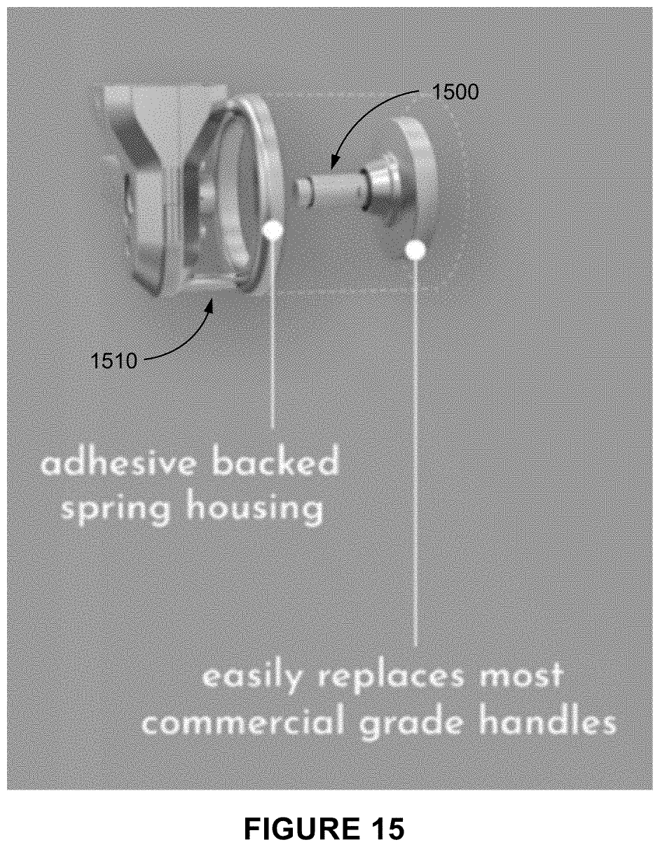

FIG. 15 depicts a sanitizing door handle adapter configured to replace an existing door handle according to various implementations of the invention. As depicted in FIG. 15, sanitizing door handle 100 may be configured to mount to a spindle 1500 of an existing latch mechanism (not otherwise illustrated) of a door, thereby replacing an existing door handle. In some implementations, sanitizing door handle 100 may be mounted to spindle 1500 by an adaptor 1510 as would be appreciated. In some implementations, adaptor 1510 may include a spring to assist with return rotation of sanitizing door handle 100 after operation as would be appreciated. In some implementations of the invention, adaptor 1510 may be adjustable or vary in size to fit a range of existing door handles. Sanitizing door handle 100 may be configured to replace either, or both, left-opening door handles or right-opening door handles as would be appreciated.

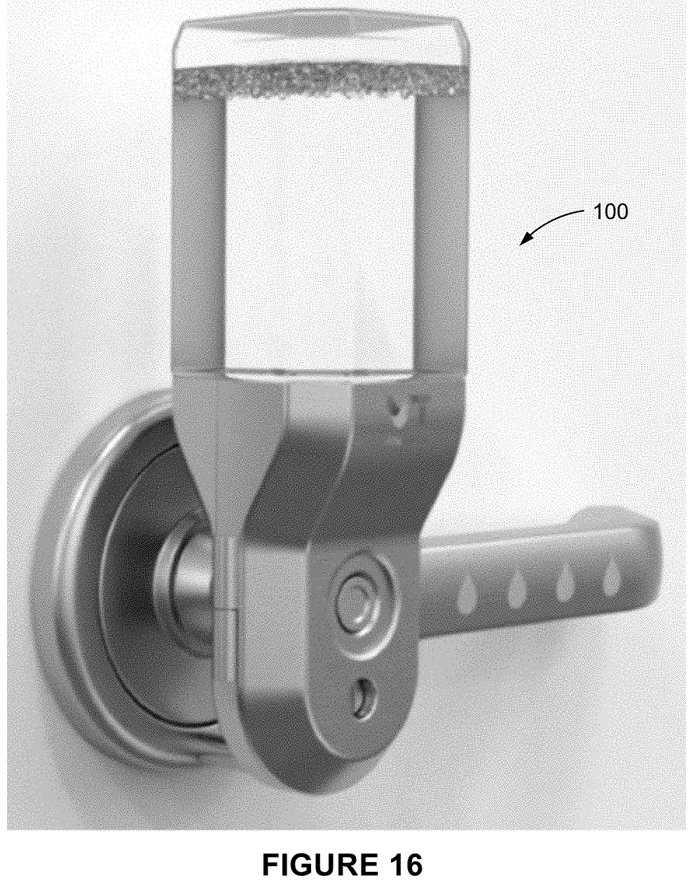

FIG. 16 depicts a sanitizing door handle according to various implementations of the invention. As depicted in FIG. 16, sanitizing door handle 100 replaces an existing door handle in its entirety according to various implementations of the invention. In some implementations, sanitizing door handle 100 replaces an existing door handle by mounting to an existing spindle and latch mechanism such as described above with reference to FIG. 15. In some implementations, sanitizing door handle 100 may be used in place of a conventional door handle. In some implementations, sanitizing door handle 100 may be configured as either, or both, a left-opening door handle or a right-opening door handle as would be appreciated.

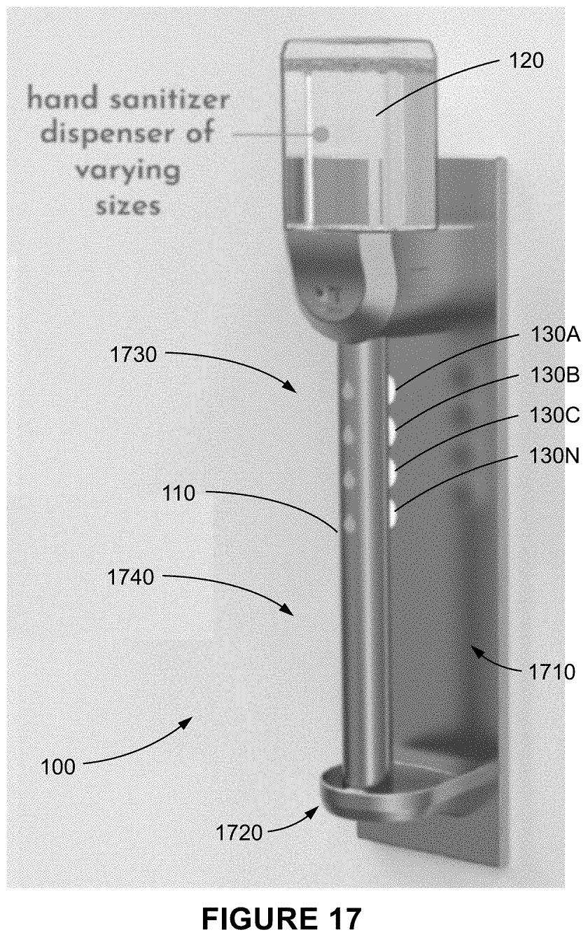

FIG. 17 depicts a sanitizing door handle according to various implementations of the invention. As depicted in FIG. 17, sanitizing door handle 100 is configured as a door handle pull according to various implementations of the invention. Sanitizing door handle 100 comprises fluid container 120 and door handle housing 110 including a plurality of dispensers 130. In some implementations of the invention, sanitizing door handle 100 includes an optional door plate 1710 and/or an optional drip tray 1720. As depicted, in some implementations, door handle housing 110 may include a first portion 1730 in which dispensers 130 are disposed, and a second portion 1740 without any dispensers. In such implementations, users may selectively grasp (or engage) either first portion 1730 (and subsequently contact dispensers 130 and the fluid) or second portion 1740 as would be appreciated. As depicted, dispensers 130 may be disposed facing the door, though other orientations of dispensers may be used as would be appreciated.

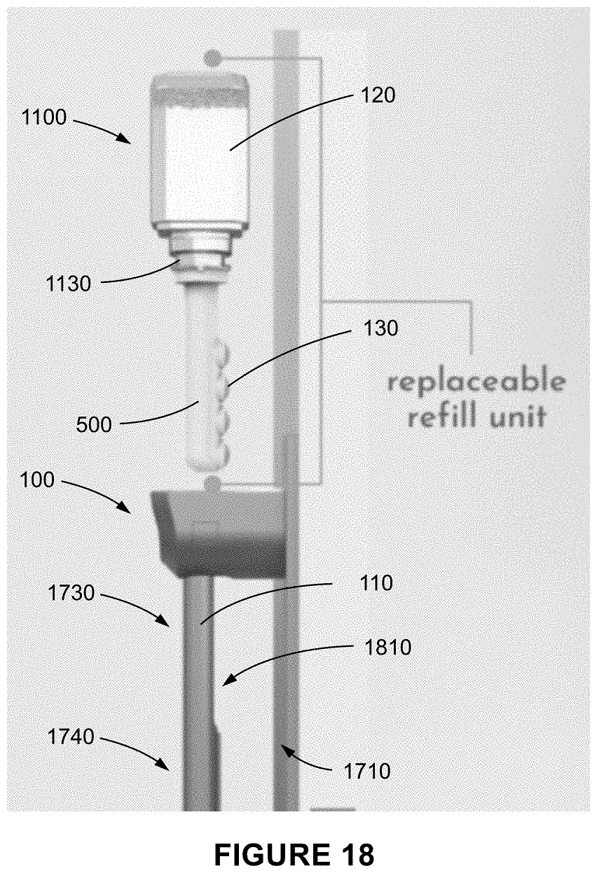

FIG. 18 depicts a replaceable refill unit configured to use with a sanitizing door handle according to various implementations of the invention. In FIG. 18, replaceable refill unit 1100 is depicted as displaced from door handle 100 to illustrate its removal/installation vis-à-vis door handle 100. As depicted, replaceable refill unit 1100 comprises fluid container 120, coupler 1130, and distribution manifold 500 including a plurality of dispensers 130 disposed therein according to various implementations of the invention. In some implementations, replaceable refill unit 1100 may include fewer or additional components as described above. In some implementations, door handle 100 is configured to removably receive replaceable refill unit 1100 within door handle housing 110. In some implementations, when in place in door handle 100, replaceable refill unit 1100 resides within door handle housing 110. In some implementations, when in place in door handle 100, at least distribution manifold 500 resides within door handle housing 110. In some implementations, door handle housing 110 includes a recess 1810 (corresponding to first portion 1730) that exposes dispensers 130 when replaceable refill unit 1100 is installed in door handle housing 110.

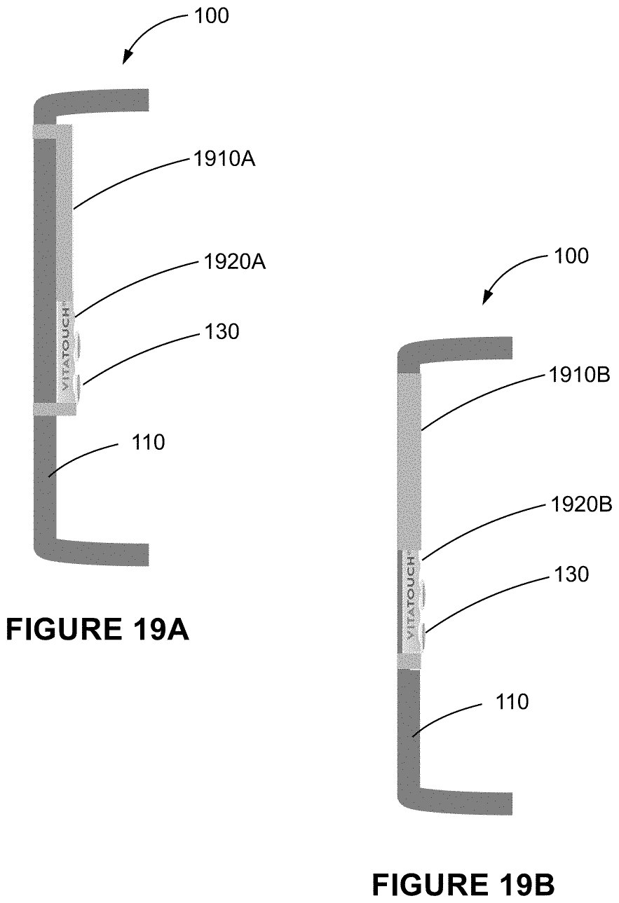

FIGS. 19A and 19B depict sanitizing door handles according to various implementations of the invention. More specifically, FIG. 19A depicts a “clip-on” adaptor 1910A that attaches to a vertical door handle 100 in accordance with various implementations of the invention. In some implementations of the invention, adaptor 1910A is configured to receive a sanitizing door handle cassette 1920A, which in turn is configured to receive distribution manifold 500, including one or more dispensers 130. In some implementations of the invention, adaptor 1910A is configured to receive cassette 1920A, which is itself distribution manifold 500. Various configurations of adaptor 1910A may be used to attach or affix cassette 1920A to door handle 100 as would be appreciated. FIG. 19B depicts an adaptor 1910B that is originally manufactured (or modified) as part of door handle 100 to receive a sanitizing door handle cassette 1920B in accordance with various implementations of the invention. In some implementations of the invention, adaptor 1910B is configured to receive cassette 1920B, which is itself distribution manifold 500. In some implementations, adaptor 1910B (or its function) may be formed into and/or part of door handle 100 as would be appreciated.

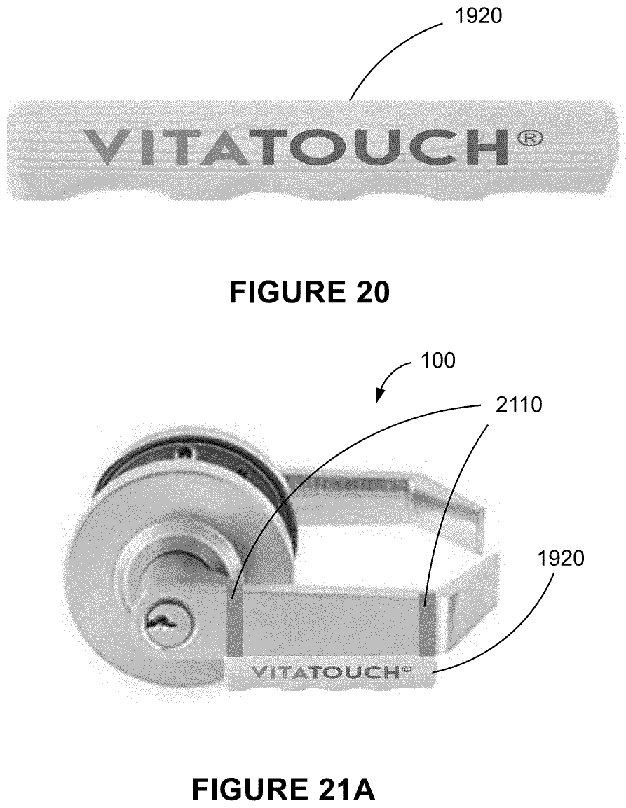

FIG. 20 depicts a sanitizing door handle cassette 1920 according to various implementation of the invention. According to various implementations of the invention, cassette 1920 comprises one or more dispenser 130 and/or a fluid container 120. In some implementations, cassette 1920 may be a single use cassette (i.e., disposable once fluid in fluid container is depleted. In some implementations, cassette 1920 may be refillable by a factory or recycle center as would be appreciated. In some implementations, cassette 1920 may be refillable at a point of use as would be appreciated.

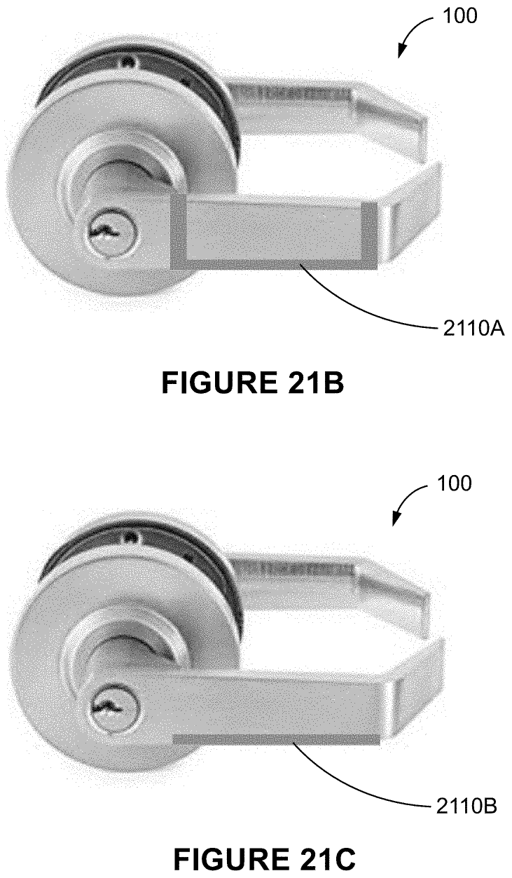

FIG. 21A depicts a sanitizing door handle according to various implementations of the invention. More specifically, FIG. 21A depicts a “clip-on” adaptor 2110 that attaches to horizontal door handle 100 in accordance with various implementations of the invention. In some implementations of the invention, adaptor 2110 is configured to receive sanitizing door handle cassette 1920, which in turn is configured to receive distribution manifold 500, including one or more dispensers 130. In some implementations of the invention, adaptor 2110 is configured to receive cassette 1920, which is itself distribution manifold 500. Various configurations of adaptor 2110 may be used to attach or affix cassette 1920 to door handle 100 as would be appreciated.

FIG. 21B depicts clip-on adaptor 2110A without cassette 1920 in accordance with various implementations of the invention. FIG. 21C depicts an adaptor 2110B 1920, where adaptor 2110B is originally manufactured (or modified) as part of door handle 100 to receive sanitizing door handle cassette 1920 in accordance with various implementations of the invention. In some implementations of the invention, adaptor 2110B is configured to receive cassette 1920, which is itself distribution manifold 500. In some implementations, adaptor 2110B (or its function) may be formed into and/or part of door handle 100 as would be appreciated.

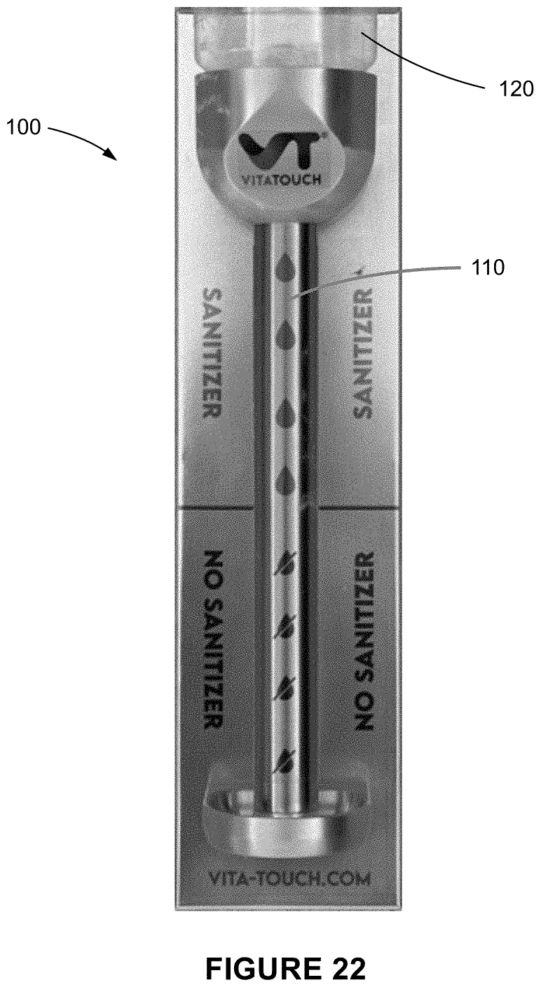

FIG. 22 depicts a sanitizing door handle 100 according to various implementations of the invention. Door handle 100 may include a fluid container 120 (only the bottom portion of which is depicted in FIG. 22). In some implementations of the invention, fluid container 120 is cylindrical in shape to facilitate installation. For example, fluid container 120 may be threaded into door handle housing 110 or into distribution manifold 500 (not illustrated in FIG. 22) which may be disposed in door handle housing 110 as would be appreciated. As depicted, door handle 100 has an upper portion that dispenses sanitizing fluid in accordance with various implementations of the invention, and a lower portion that does not dispense sanitizing fluid. In some implementations of the invention, distribution manifold 500 occupies the upper portion of door handle housing 110 to facilitate dispensing of sanitizing fluid.

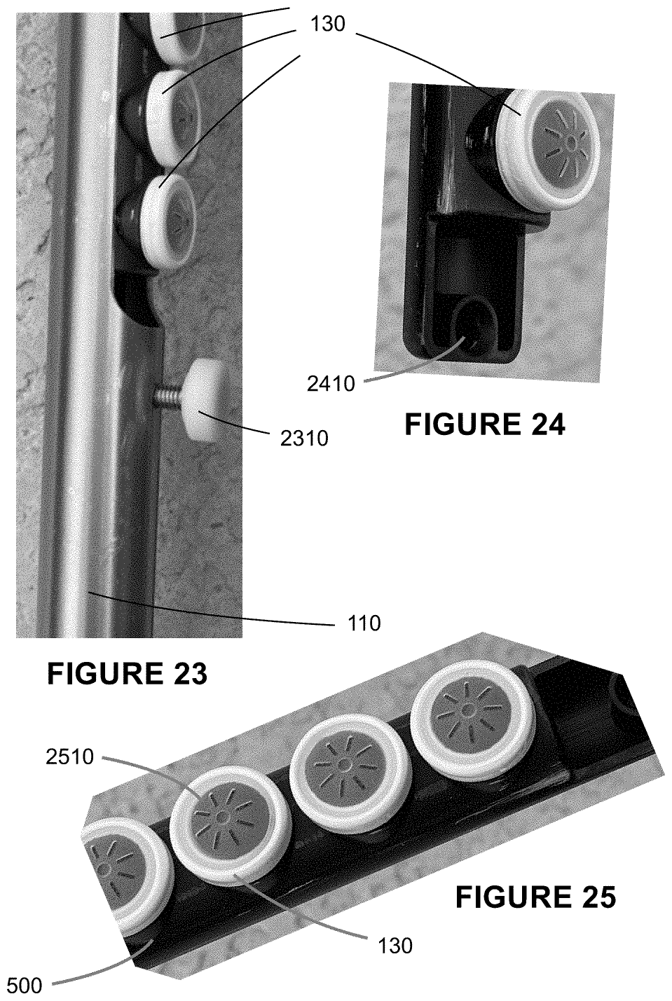

FIG. 23 depicts a distribution manifold 500 partially installed in a door handle housing 110 according to various implementations of the invention. FIG. 24 depicts a retaining portion 2410 of distribution manifold 500 according to various implementations of the invention. In some implementations of the invention, a screw 2310 is inserted through door handle housing and into retaining portion 2410 of distribution manifold 500 to secure distribution manifold 500 in place. Other mechanisms for retaining distribution manifold 500 in door handle housing 110 may be used, including snap fit, retaining clips, etc., as would be appreciated.

FIG. 25 depicts dispensers 130 with perforated plastic membranes 2510 according to various implementations of the invention. As depicted, membranes 2510 include one or more perforations which allow fluid to flow through plastic membrane 2510 as would be appreciated. Various numbers of perforations, patterns, sizes, etc., may be used to control fluid flow through plastic membrane 2510 as would be appreciated. In some implementations, membrane 2510 may be comprised of a non-porous or semi-porous material other than plastic, including but not limited to, metal, rubber, ceramic, etc., as would be appreciated; these other non-porous or semi-porous materials may be perforated as would be appreciated.

While various implementations of the invention have been described herein in reference to certain door handles illustrated in the drawings, for example, other door handles and configurations thereof may be used as would be appreciated.

While various implementations of the invention have been described herein in reference to door handles and doors, other implementations of the invention may be configured for use with barriers other than doors, such as but not limited to, gates, pass-throughs, security checkpoints, or other barriers, where users regularly contact a particular surface for access through or past the barrier.

What is claimed:

1. A replaceable refill unit comprising:
a fluid container configured to retain a sanitizing fluid; and
a distribution manifold having at least one dispenser disposed therein, the distribution manifold configured to distribute the sanitizing fluid from the fluid container to the dispenser, the dispenser configured to dispense the sanitizing fluid.

2. The replaceable refill unit of claim 1, further configured to be disposed within or affixed to a door handle.

3. The replaceable refill unit of claim 1, wherein the sanitizing fluid is gravity-fed from the fluid container to the distribution manifold.

4. The replaceable refill unit of claim 1, wherein the fluid container and the distribution manifold are removably coupled to one another.

5. The replaceable refill unit of claim 1, wherein the fluid container and the distribution manifold are affixed to one another.

6. The replaceable refill unit of claim 1, wherein the at least one dispenser comprises a plurality of dispensers.

7. The replaceable refill unit of claim 1, wherein each dispenser comprises a spring configured to prevent flow of the sanitizing fluid out of the dispenser when in a first compressed state and to dispense the sanitizing fluid when further compressed by a user.

8. The replaceable refill unit of claim 1, wherein the fluid container is refillable.

9. The replaceable refill unit of claim 1, wherein the distribution manifold is configured to be disposed within a door handle.

10. The replaceable refill unit of claim 1, wherein the distribution manifold is configured to be disposed within a door handle such that the door handle provides a sanitizing portion and a non-sanitizing portion.

11. The replaceable refill unit of claim 1, wherein each dispenser comprises a liquid permeable membrane through which the sanitizing liquid is dispensed.

12. The replaceable refill unit of claim 1, wherein each dispenser comprises a perforated plastic membrane through which the sanitizing liquid is dispensed.

13. The replaceable refill unit of claim 1, further comprising an adaptor configured to removably affix the replaceable refill unit to an exterior of a door handle.

14. The replaceable refill unit of claim 1, further comprising an adaptor configured to removably affix the replaceable refill unit to an interior of a door handle.

15. An apparatus comprising:
a distribution manifold configured to be affixed to or disposed within a door handle and configured to removably attach to a sanitizing fluid container, wherein the distribution manifold comprises at least one dispenser, wherein the distribution manifold is further configured to distribute a sanitizing fluid from the sanitizing fluid container to the at least one dispenser via gravity, wherein the dispenser operatively dispenses the sanitizing fluid to a user.

16. The apparatus of claim 15, wherein the at least one dispenser comprises a spring configured to prevent flow of the sanitizing fluid out of the at least one dispenser when in a first compressed state and to dispense the sanitizing fluid when further operatively compressed by a user.

17. The apparatus of claim 15, wherein the at least one dispenser comprises a liquid permeable membrane through which the sanitizing liquid is dispensed.

18. The apparatus of claim 15, wherein the at least one dispenser comprises a perforated plastic membrane through which the sanitizing liquid is dispensed.
